(19) United States
(12) Patent Application Publication
(10) Pub. No.: US 2008/0021328 A1
(43) Pub. Date: Jan. 24, 2008

(54) Ultrasound probe and method of manufacturing ultrasound probe

(75) Inventors: Takeshi Habu, Tokyo (JP); Takayuki Sasaki, Tokyo (JP)

Correspondence Address: FRISHAUF, HOLTZ, GOODMAN & CHICK, PC, 220 Fifth Avenue, 16TH Floor, NEW YORK, NY, 10001-7708, US

(73) Assignee: KONICA MINOLTA MEDICAL & GRAPHIC, INC., Tokyo (JP)

(21) Appl. No.: 11/825,744

(22) Filed: Jul. 9, 2007

(30) Foreign Application Priority Data
Jul. 20, 2006 (JP) JP2006-197839

(51) Int. Cl. A61B 8/00

(52) U.S. Cl. 600/459

(57) ABSTRACT

An objective is to provide a multichannel array type ultrasound probe possessing a transmission-reception separation type composite piezoelectric element, in which the operation during transmission and reception of ultrasound wave is separated, wherein a piezoelectric element for transmission possesses a ceramic material, a piezoelectric element for reception possesses a high sensitivity organic piezoelectric element material, and an acoustic matching layer is provided between a piezoelectric element for transmission and a piezoelectric element for reception to enhance high sensitivity in S/N ratio. Also disclosed is a multichannel array type ultrasound probe possessing a transmission-reception separation type composite piezoelectric element, wherein the composite piezoelectric element comprises an acoustic matching layer provided between a piezoelectric element for transmission possessing a ceramic material and a piezoelectric element for reception possessing an organic material.

This application claims priority from Japanese Patent Application No. 2006-197839 filed on Jul. 20, 2006, which is incorporated hereinto by reference.

### TECHNICAL FIELD

The present invention relates to an array type ultrasound probe utilized for medical diagnosis, and a manufacturing method thereof.

### BACKGROUND

An ultrasound diagnosis apparatus is a medical imaging apparatus which obtains a tomographic image of a soft tissue in a biological object via an ultrasound pulse reflection method with no damage. This ultrasound diagnosis apparatus is compact, inexpensive and safe with no exposure to X-rays, in comparison to other medical imaging apparatuses, and is capable of perfusion imaging by applying Doppler effect. Further, the ultrasound diagnosis apparatus is widely utilized in departments of cardiovascular disease (arteria coronaria of the heart), digestive organs (gastrointestinal), internal medicine (liver, spleen and pancreas), urology (kidney and bladder), obstetrics and gynecology. The piezoelectric effect of a piezoelectyric ceramic is utilized for an ultrasound probe employed for such the ultrasound medical diagnosis apparatus to perform ultrasonic transmission and reception at high resolution. In this case, as a vibration mode of the piezoelectric element for transmission and reception, a single type prove and an array type probe in which a plurality of probes are placed two-dimensionally are generally used. Since the array type can obtain fine images, it is widely utilized for medical images for diagnostic examination

On the other hand, since harmonic imaging diagnosis employed with a harmonic signal can obtain a clear image which can not be acquired by conventional B mode diagnosis, it is to be employed as a standard diagnosis modality.

The harmonic imaging has various advantages in that a contrast resolution is improved because of a superior S/N ratio due to a small side lobe level, a horizontal resolution is improved since a beam width becomes thin with an increase of frequency, multireflection is not generated because an acoustic pressure is small at a short distance, fluctuation of the acoustic pressure is also small, attenuation in the range beyond a focal point is at the same level as that of a fundamental wave, and a larger depth-extending speed can be taken, compared with an ultrasound wave in which the frequency of the harmonic wave is based on the fundamental wave.

As a specific structure of the array type ultrasound probe for the harmonic imaging, a piezoelectric transducer in which each transducer element constituting an array is a broad band integrated type is employed. There is generally utilized a method in which the fundamental wave is transmitted in a frequency range on the low frequency side of a broad band characteristic, and the harmonic wave is received in a frequency range on the high frequency side. Under such the situation, concerning a conventional ultrasound probe, a technique to improve sensitivity is known (refer to Patent document 1). Herein, a transducer in which a fine columnar structure piezoelectric element is fixed with an organic compound such as an epoxy resin, for example, is used as an ultrasound wave transmission-reception element, and each columnar structure ceramic is longitudinally oscillated to improve sensitivity.

A narrow band ultrasound wave is employed so as not to overlap a spectrum of a fundamental wave-transmitting ultrasound wave and a spectrum of a harmonic wave-receiving ultrasound wave as much as possible. However, since the narrow band ultrasound wave is generally an ultrasound pulse signal having a so-called long tail, resolution in a depth direction is deteriorated.

As the other specific structure of the array type ultrasound probe for harmonics imaging, disclosed is a transmission-reception separation type probe in which each of a piezoelectric transducer for transmission and a piezoelectric transducer for reception is independently placed (refer to Patent Documents 1 and 2). For example, as for transmission of the fundamental wave and reception of the ultrasound wave including a harmonic wave, it is also disclosed that provided are the first piezoelectric layer composed of a plurality of the first arrayed piezoelectric elements having the first acoustic impedance to carry the function of transmission-reception of an ultrasound wave having a center frequency of f₁, and the second piezoelectric layer composed of a plurality of the second arrayed piezoelectric elements having the second acoustic impedance, which is superimposed on the first piezoelectric layer, to carry the function of reception of an ultrasound wave having a center frequency of f₂=2·f₁ (refer to Patent Document 2), but sufficient sensitivity has not yet been acquired.

Further, for the purpose of improving sensitivity, in order to produce an ultrasound wave transmission-reception element, a piezoelectric ceramic element is laminated to lower an apparent impedance and to improve an electrical matching condition with a driving circuit, and an electric field intensity is increased to generate a large distortion, whereby transmission sensitivity is improved. However, in the case of the laminated structure, transmission sensitivity is increased in accordance with the number of laminated layers, but reception sensitivity is inversely proportional to the number of laminated layers. As a result, the harmonic imaging is to be at a disadvantage.

(Patent Document 1) Japanese Patent O.P.I. Publication No. 8-187245

(Patent Document 2) Japanese Patent O.P.I. Publication No. 11-276478

### SUMMARY

The present invention was made on the basis of the above-described situation. It is an object of the present invention to provide a multichannel array type ultrasound probe comprising a transmission-reception separation type composite piezoelectric element, in which the operation during transmission and reception of ultrasound wave is separated, wherein a piezoelectric element for transmission comprises a ceramic material, a piezoelectric element for reception comprises a high sensitivity organic piezoelectric element material, and an acoustic matching layer is provided between a piezoelectric element for transmission and a piezoelectric element for reception to enhance high sensitivity in S/N ratio.

In order to solve the foregoing problem, as for an ultrasound probe of the present invention, a piezoelectric element for transmission having a structure in which a piezoelectric ceramic constituting each channel is single-layered or laminated, and a piezoelectric element for reception which is single-layered or laminated are separated into piezoelectric elements for transmission and reception, and at the same time, an acoustic matching layer is provided between the piezoelectric element for transmission and the piezoelectric element for reception to obtain a high sensitivity ultrasound probe.

### BRIEF DESCRIPTION OF THE DRAWINGS

Embodiments will now be described, by way of example only, with reference to the accompanying drawings which are meant to be exemplary, not limiting, and wherein like elements numbered alike in several figures, in which:

FIG. 1 is a schematic cross-sectional view showing an example of a conventional type ultrasound prove, and

FIG. 2 is a schematic cross-sectional view showing an example of a transmission-reception separation type ultrasound prove in the embodiments of the present invention.

### DESCRIPTION OF THE PREFERRED EMBODIMENTS

The above object of the present invention is accomplished by the following structures.

(Structure 1) A multichannel array type ultrasound probe comprising a transmission-reception separation type composite piezoelectric element, wherein the composite piezoelectric element comprises an acoustic matching layer provided between a piezoelectric element for transmission comprising a ceramic material and a piezoelectric element for reception comprising an organic material.

(Structure 2) The multichannel array type ultrasound probe of Structure 1, wherein the organic material comprises a resin selected from the group consisting of polyvinylidene fluoride, polyurea, polyamide, polyimide, polyester and polyolefin in an amount of 60-100 mol %.

(Structure 3) The multichannel array type ultrasound probe of Structure 1, wherein the organic material further comprises a vinylidene fluoride-perfluoroalkylvinyl ether copolymer or a vinylidene fluoride-perfluoroalkoxyethylene copolymer, provided that a content of the vinylidene fluoride is 85-99 mol %, and a content of the perfluoroalkylvinyl ether or the perfluoroalkoxyethylene is 1-15 mol %.

(Structure 4) The multichannel array type ultrasound probe of Structure 1, wherein the acoustic matching layer comprises plural layers, and an acoustic impedance value of each of both outermost layers among the plural layers is 5-28 Mrayl.

(Structure 5) The multichannel array type ultrasound probe of Structure 1, wherein the acoustic matching layer comprises a plurality of layers each having a different filler content.

(Structure 6) The multichannel array type ultrasound probe of Structure 5, wherein the filler is least one selected from the group consisting of tungsten, ferrite and aluminum.

(Structure 7) The multichannel array type ultrasound probe of Structure 1, wherein the ceramic material comprises one selected from the group consisting of PZT, quartz, lithium niobate (LiNbO₃), potassium niobate tantalate [K(Ta,Nb)O₃], barium titanate (BaTiO₃), lithium tantalate (LiTaO₃) and strontium titanate (SrTiO₃).

(Structure 8) The multichannel array type ultrasound probe of Structure 1, wherein the acoustic matching layer comprises a binder selected from the group of consisting of polyvinyl butyral, polyolefin, polycycloolefin, polyacrylate, polyamide, polyimide, polyester, polysulfone, silicone, epoxy and derivatives thereof.

(Structure 9) The multichannel array type ultrasound probe of Structure 1, wherein the transmission-reception separation type composite piezoelectric element comprises the piezoelectric element for transmission and the piezoelectric element for reception, provided between an acoustic lens and a backing layer.

(Structure 10) A method of manufacturing the multichannel array type ultrasound probe, comprising the step of coating the acoustic matching layer of Structure 1 via ink-jet.

(Structure 11) The method of Structure 10, further comprising the step of discharging a liquid comprising a binder or a filler as ink used for the ink-jet.

While the preferred embodiments of the present invention have been described using specific terms, such description is for illustrative purposes only, and it is to be understood that changes and variations may be made without departing from the spirit or scope of the appended claims.

### DETAILED DESCRIPTION OF THE INVENTION

The preferred embodiments of the present invention will be explained below, but the present invention is not limited thereto.

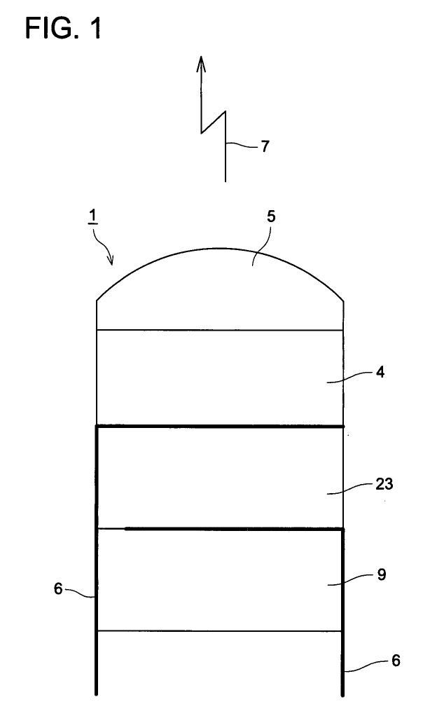

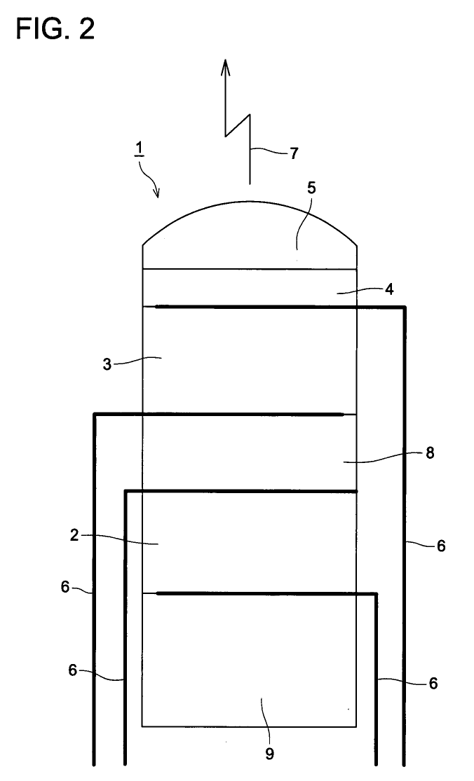

Next, the embodiments of the present invention will be described referring to FIG. 1 and FIG. 2.

FIG. 1 is a schematic cross-sectional view showing an example of a conventional type ultrasound prove.

FIG. 2 is a schematic cross-sectional view showing an example of a transmission-reception separation type ultrasound prove in the embodiments of the present invention.

Ultrasound probe 1, transmission-reception integrated type piezoelectric element 23 (a piezoelectric element for transmission and a piezoelectric element for reception are installed), piezoelectric element for transmission 2, piezoelectric element for reception 3, acoustic matching layer 4 (which may be present or absent), acoustic lens 5, each electrode 6, transmitting direction of ultrasound wave 7, acoustic matching layer of the present invention 8 and backing material 9 are shown in FIG. 1 and FIG. 2.

An example of a method of manufacturing an ultrasound probe in the embodiments of the present invention will be described below, referring to FIG. 2.

First, piezoelectric element for transmission 2 is prepared on backing material 9. As shown in FIG. 2, a transmission-reception separation type composite piezoelectric element has a laminated structure in which acoustic matching layer 8 (the present invention) is provided between piezoelectric element for transmission 2 and piezoelectric element for reception 3. Piezoelectric element for transmission 2 may have a structure in which s thin piezoelectric plate and an electrode layer are interleaved as shown in FIG. 2. Such the structure can be produced by laminating a piezoelectric ceramic green sheet on which an electrode is printed employing silver paste before calcinations, and sintering the resulting entirely. The thickness of a green sheet can be adjusted, depending on oscillation frequency.

In the present invention, at least one acoustic matching layer 8 is placed on piezoelectric element for transmission 2. A plurality of acoustic matching layers in the range of 2-5 layers can be provided by filling a filler in a polymerized resin, and a filling amount of filler may be varied so as to produce a gradient material. A stereolithography resin may be cured by exposing it to laser light via a method of varying a filling amount of filler as described in Japanese Patent O.P.I. Publication No. 2003-169397, and the filler concentration may be varied while discharging a resin material and a filler via ink-jet. When it is difficult to dissolve a resin, coating the acoustic matching layer is preferably conducted by discharging oligomer or a monomer raw material from an ink-jet head.

Piezoelectric element for reception 3 may be attached with a previously prepared plate or sheet employing an adhesive similarly to piezoelectric element for transmission 2, and a resin may be coated onto an acoustic matching layer and dried to form a film. A thin film can also be formed by discharging a resin raw material monomer or oligomer from an ink-jet head, and can further be formed via thermal polymerization, or film formation by polymerization employing light, X-ray or electron beam after coating a monomer. Silver paste, platinum paste, palladium paste or such may be employed for an electrode.

The ultrasound probe of the present invention may comprise an acoustic matching layer in which cured layers made of a photo-curing resin or a thermosetting resin are laminated, on a piezoelectric element. In this case, since the ultrasound probe can be formed without attaching an acoustic matching layer onto a piezoelectric element, prepared can be the acoustic matching layer in which no bubbles generated from use of an adhesive, and less fluctuation in characteristics is exhibited.

Further, the ultrasound probe of the present invention preferably comprises an acoustic matching layer in which any filler is mixed in a photo-curing resin or a thermosetting resin. In this case, easily prepared can be the acoustic matching layer having an adequate acoustic impedance value to perform ultrasound wave transmission and reception efficiently. As to the ultrasound probe of the present invention, the acoustic matching layer may have a different acoustic impedance value gradually in the thickness direction. An ultrasound wave signal for transmission and reception from a piezoelectric element can be efficiently transmitted when the acoustic matching layer has a laminated layer composed of a plurality of layers which have different filler contents each sequentially, and the acoustic matching layer having sequentially different acoustic impedance values is formed.

An acoustic matching layer formed from an admixture of at least two kinds of fillers having different particle diameters, or a photo-curing resin or a thermosetting resin incorporating different fillers in the thickness direction is preferably prepared for an ultrasound probe of the present invention. In this case, an acoustic impedance value of the acoustic matching layer to perform ultrasound wave transmission and reception efficiently can be adequately adjusted, whereby the ultrasound wave signal of a piezoelectric element can be efficiently transmitted and received.

Further, an acoustic matching layer formed from an admixture of at least two kinds of fillers having different densities, or a photo-curing resin or a thermosetting resin incorporating different fillers in the thickness direction can be prepared for an ultrasound probe of the present invention. This can transmit and receive the ultrasound wave signal of a piezoelectric element efficiently.

As to the ultrasound probe of the present invention, at least one of tungsten, ferrite and alumina is contained in a photo-curing resin as the filler. This can easily form an acoustic matching layer having an intermediate value between an acoustic impedance value of a piezoelectric element and an acoustic impedance value of a human body to transmit and receive the ultrasound wave signal of a piezoelectric element efficiently. In the above description, ferrite means a crystalline structure having iron (Fe). A filling amount of a filler with respect to a resin is preferably 0.001-20 times in quantity. A plurality of resin layers constituting the foregoing acoustic matching layer are formed, and density of the lower resin layer is designed to be larger and density of the superficial resin layer is designed to be lower, whereby the different acoustic impedance value in the thickness direction is preferably obtained. Preferable is a gradient technology in which a different acoustic impedance value in the thickness direction is obtained by making a larger amount of filler in the lower layer and a lower amount of filler in the superficial layer, utilizing a sedimentation rate of the filler contained in the foregoing resin. Further, the density of the foregoing resin layer is preferably controlled by varying at least one of an addition amount of filler, an average particle diameter and a utilized material density.

Examples of preferable thermosetting resins to fill a filler include a type in which an urethane acrylate based resin, an epoxy acrylate based resin, an ester acrylate based resin, an acrylate based resin or such is cured via radical polymerization, and a type in which an epoxy based resin, a vinyl ether based resin or such is cured via cationic polymerization. What type of resin is employed depends on reaction speed, strain with contraction, dimensional accuracy, heat resistance, strength and so forth. The urethane acrylate based resin and the epoxy based resin are mainly used as the resin, but the urethane acrylate based resin has a feature in which the reaction speed is fast, the intermolecular cohesion is large, and the mechanical and thermal strength is advantageous in comparison to the epoxy based resin, and is particularly preferable in the case of focusing on strength. On the other hand, the epoxy based resin has a feature in which the reaction speed is low, the strain with contraction is small. Therefore, and an epoxy based stereolithography resin is advantageous in view of dimensional accuracy, and is particularly preferable in the case of focusing on accuracy.

In the case of laminating the above-described layers and placing an electrode, an organic binder may not be employed to fix the electrode, but a generalized adhesive may be used in the case of being employed as a simplified method. Particularly when a ceramic piezoelectric element for transmission, an acoustic matching layer and an organic piezoelectric element for reception are incorporated, an organic binder is preferably used, since insufficient is adhesive strength between each of electrodes, between which the acoustic matching layer is put, and an interface of the acoustic matching layer, as well as adhesive strength between each of electrodes, between which an organic piezoelectric element is put, and an interface of the organic piezoelectric element, whereby peeling-off is generated. Preferable binders can be provided as shown below.

Examples thereof include resins made of polyvinyl butyral, polyolefin, polycycloolefin, Polyacrylate, polyamide, polyimide, polyester, polysulfone, silicone, epoxy and derivatives thereof. From an existing chemical substance described in the chemical substance control law, (6)-708 (CASNo.63148-65-2) alkyl(C4)acetalpolyvinylalcohol can be provided as a typical example for polyvinyl butyral. Examples of polyamide include polyamide 6, polyamide 66, polyamide 610, polyamide 612, polyamide MXD6, polyamide 11, polyamide 12, polyamide 46, methoxylated polyamide (existing chemical substance No. (7)-383) polycondensates of polyalkylen(C3)polyamine/polyalkylene(C4)dicarboxylicacid/urea and so forth. Existing chemical substance No. (7)-2211 (CASNo.611-79-0) which has been developed in NASA is provided as polyi,ide. Examples of silicone include existing chemical substance Nos. (7)-476 polyalkyl(C12)siloxane, (7)-474 polyalkyl(C12)siloxane, (7)-477 polyalkyl(hydrogen)siloxane, (7)-483 polyalkyl(C9)alkeny(C4)siloxane, and (7)-485 polyalsodiumalkyl(C4)siliconate. As an epoxy compound, there are a polyphenol based epoxy compound, a polyglycidylamine based epoxy compound, an alcohol based epoxy compound or an ester based epoxy compound, but a cycloaliphatic based epoxy compound is particularly preferable, and existing chemical substance Nos. 3-2452 3,4-epoxycyclohexylmethyl(3,4-epoxy)cyclohexanecalboxylate, 3-3453, 4-47 2-(3,4-epoxy)cyclohexyl-5,1-spiro-(3,4-epoxy)cyclohexyl-m-dioxane and 5-1052 1,3,5-tris-glycidyl-isocyanuric acid for example, are preferable.

A consumption amount of the foregoing resin to be determined depends on a frequency characteristic and sensitivity, and in terms of thickness, a thickness of 10 nm-60 μm is desired and a thickness of 20 nm-30 μm is preferable.

As to a method of using a resin, the resin may be dissolved in a solvent such as DMSO, DMF or DME, or may be thermally dissolved by heating to a melting temperature of the bulk without using a solvent.

As to a method of using a binder, the binder can be may be used for any layer in the case of laminating elements, but is preferably employed to bond a piezoelectric element for transmission and a piezoelectric element for reception. When an electrode has already been formed in the piezoelectric element for transmission via printing or coating, a binder is preferably applied on the piezoelectric element for reception in which no electrode is printed.

In the above-described process of laminating layers, any one of the two electrodes is printed in the situation where piezoelectric element for transmission 2 and piezoelectric element for reception 3 are in the form of a ceramic sheet and of an organic film sheet, respectively, and an acoustic matching layer is put between the sheets and attached together for integrated combination. In this case, taking into consideration a transmission-reception sensitivity characteristic and driving of a piezoelectric element material, or an input/output impedance of a receiving circuit, it is desired to produce piezoelectric elements for transmission and reception via selection of the thickness and material of each laminated structure. Accordingly, it is preferable that the value of impedance is appropriately selected for each of piezoelectric element for transmission 2, acoustic matching layer 8 and piezoelectric element for reception 3. Only piezoelectric element for transmission 3 is also produced via calcination with a lamination process of a green sheet, and acoustic matching layer 8 and piezoelectric element for reception 3 may be subsequently attached together to conduct coating, or in the case of vinylidene fluoride, a structure in which a laminated uniaxially stretched sheet which is coated and dried in advance (organic piezoelectric sheet) is employed and adhered may be utilized. Specifically preferable is a lamination type in which vinylidene fluoride based sheet is uniaxially stretched in advance so as to maximize the piezoelectric effect, and a sheet subjected to a poling treatment is attached employing an organic binder.

A vinylidene fluoride/trifluoroethylene copolymer used for a polymeric piezoelectric film having low tensile modulus is particularly preferred as the organic piezoelectric sheet. The sheet is obtained by increasing a slow cooling speed to approximately 3° C./minute during a heat treatment process after film formation (process of enhancing crystallinity by applying a temperature between a ferroelectric-paraelectric phase transition point and a melting point), and further, tensile modulus can slightly be lowered by annealing at 100° C. for several 10 minutes (20-30 minutes) after conducting a poling treatment of a piezoelectric film. Any of other methods may also be used, provided that tensile modulus is lowered by handling during a manufacturing process.

As for a molecular weight of a raw material polymer, a polymer generally exhibits plasticity and flexibility with increasing a molecular weight, resulting in a piezoelectric film having low tensile modulus. In the case of P (VDF-TrFE) and/or (VDF-TeFE), a polymeric piezoelectric film having low tensile modulus is realized by employing a polymeric piezoelectric film having a melt flow rate of at most 0.02 g/minute at 230° C., or preferably at most 0.01 g/minute, whereby a high sensitivity piezoelectric sheet can be obtained. The above-described VDF represents vinylidene fluoride, TrFE represents trifluoroethylene, and TeFE represents tetrafluoroethylene.

On the other hand, in the case of vinylidene fluoride/trifluoroethylene, since an electromechanical coupling factor (piezoelectric effect) in the thickness direction depending on a copolymerization ratio varies, the former in the copolymerization ratio is preferably 60-99 mol %, but since it also depends on a method of using an organic binder employed when a ceramic piezoelectric element and an organic piezoelectric element are superimposed, the optimal value also varies. The most preferable value of the foregoing copolymerization ratio is 85-99 mol %.

A polymer having 85-99 mol % of vinylidene fluoride and 1-15 mol % of perfluoroalkylvinyl ether, perfluoroalkoxyethylene or perfluorohexaethylene is particularly preferable since sensitivity of a harmonic wave reception is enhanced by suppressing the fundamental wave of transmission in the case of a combination of a ceramic piezoelectric element for transmission and an organic piezoelectric element for reception. Tetrafluoroethylene and trifluoroethylene have conventionally been accepted so far, but perfluoroalkylvinylether (PFA), perfluoroalkoxyethylene (PAE) or perfluorohexaethylene is preferably employed for a composite element of the present invention.

Polyurea resins are provided as other polymers usable for the piezoelectric element. Polyurea is preferably one employed in combination of the following a/b. Examples thereof include 4,4′-diaminodiphenylmethane/3,3′-dimethyldiphenyl-4,4′-diisocyanate, 4,4′-diamino diphenylmethane/o-dianisidine diisocyanate, 4,4′-diaminodiphenylmethane/methylenebis(4-isocyanate-2-methylbenzene), 4,4′-diaminodiphenylmethane/4,4′-diphenylmethanediisocyanate(MDI), 4,4′-diaminodiphenylmethane/2,4-toluenediisocyanate(2,4-TDI), 4,4′-diaminodiphenylmethane/2,6-toluenediisocyanate(2,6-TDI), 4,4′-diaminodiphenylmethane/bis(4-isocyanatephenyl)ether, 4,4′-diaminodiphenylmethane/p-phenylenediisocyanate, 4,4′-diamino diphenylmethane/1,5-naphthalenediisocyanate, 4,4′-diaminodiphenylether/3,3′-dimethyldiphenyl-4,4′-diisocyanate, 4,4′-diaminodiphenylether/o-dianisidinediisocyanate, 4,4′-diaminodiphenylether/methylenebis(4-isocyanate-2-methylbenzene), 4,4′-diaminodiphenylether/4,4′-diphenylmethanediisocyanate(MDI), 4,4′-diaminodiphenylether/2,4-toluenediisocyanate(2,4-TDI), 4,4′-diaminodiphenylether/2,6-toluenediisocyanate(2,6-TDI), 4,4′-diaminodiphenylether/bis(4-isocyanatephenyl)ether, 4,4′-diaminodiphenylether/p-phenylenediisocyanate, 4,4′-diaminodiphenylether/1,5-naphthalenediisocyanate, 4,4′-diaminodiphenylether/1,3-bis(isocyanatemethyl)benzene, 4,4′-diamino-3,3′-dimethyldiphenylmethane/3,3′-dimethydiphenyl-4,4′-diisocyanate, 4,4′-diamino-3,3′-dimethyldiphenylmethane/o-dianisidinediisocyanate, 4,4′-diamino-3,3′-dimethyldiphenylmethane/methylenebis(4-isocyanate-2-methylbenzene), 4,4′-diamino-3,3′-dimethyldiphenylmethane/4,4′-diphenylmethanediisocyanate(MDI), 4,4′-diamino-3,3′-dimethyldiphenylmethane/2,4-toluenediisocyanate(2,4-TDI), 4,4′-diamino-3,3′-dimethyldiphenylmethane/2,6-toluenediisocyanate(2,6-TDI), 4,4′-diamino-3,3′-dimethyldiphenylmethane/bis(4-isocyanatephenyl)ether, 4,4′-diamino-3,3′-dimethyldiphenylmethane/p-phenylenediisocyanate, 4,4′-diamino-3,3′-dimethyldiphenylmethane/1,5-naphthalenediisocyanate, 3,3′-dimethoxy-4,4′-diaminobiphenyl/3,3′-dimethyldiphenyl-4,4′-diisocyanate, 3,3′-dimethoxy-4,4′-diaminobiphenyl/o-dianisidinediisocyanate, 3,3′-dimethoxy-4,4′-diaminobiphenyl/methylenebis(4-isocyanate-2-methylbenzene), 3,3′-dimethoxy-4,4′-diaminobiphenyl/4,4′-diphenylmethane diisocyanate(MDI), 3,3′-dimethoxy-4,4′-diaminobiphenyl/2,4-toluenediisocyanate (2,4-TDI), 3,3′-dimethoxy-4,4′-diaminobiphenyl/2,6-toluenediisocyanate (2,6-TDI), 3,3′-dimethoxy-4,4′-diaminobiphenyl/bis(4-isocyanatephenyl)ether, 3,3′-dimethoxy-4,4′-diaminobiphenyl/p-phenylenediisocyanate, 3,3′-dimethoxy-4,4′-diaminobiphenyl/1,5-naphthalenediisocyanate, 3,3′-dimethyl-4,4′-diaminobiphenyl/3,3′-dimethyldiphenyl-4,4′-diisocyanate, 3,3′-dimethyl-4,4′-diaminobiphenyl/o-dianisidinediisocyanate, 3,3′-dimethyl-4,4′-diaminobiphenyl/methylenebis(4-isocyanate-2-methylbenzene), 3,3′-dimethyl-4,4′-diaminobiphenyl/4,4′-diphenylmethanediisocyanate(MDI), 3,3′-dimethyl-4,4′-diaminobiphenyl/2,4-toluenediisocyanate(2,4-TDI), 3,3′-dimethyl-4,4′-diaminobiphenyl/2,6-toluenediisocyanate(2,6-TDI), 3,3′-dimethyl-4,4′-diaminobiphenyl/bis(4-isocyanatephenyl)ether, 3,3′-dimethyl-4,4′-diaminobiphenyl/p-phenylenediisocyanate, 3,3′-dimethyl-4,4′-diaminobiphenyl/1,5-naphthalenediisocyanate, 4,4′-methylene-bis(2-chloroaniline)/3,3′-dimethyldiphenyl-4,4′-diisocyanate, 4,4′-methylene-bis(2-chloroaniline)/o-dianisidinediisocyanate, 4,4′-methylene-bis(2-chloroaniline)/methylenebis(4-isocyanate-2-methylbenzene), 4,4′-methylene-bis(2-chloroaniline)/4,4′-diphenylmethanediisocyanate(MDI), 4,4′-methylene-bis(2-chloroaniline)/2,4-toluenediisocyanate (2,4-TDI), 4,4′-methylene-bis(2-chloroaniline)/2,6-toluenediisocyanate (2,6-TDI), 4,4′-methylene-bis(2-chloroaniline)/bis(4-isocyanatephenyl)ether, 4,4′-methylene-bis(2-chloroaniline)/p-phenylenediisocyanate, 4,4′-methylene-bis(2-chloroaniline)/1,5-naphthalenediisocyanate, and 1,3-diamino-5-cyanobenzene/2,6-naphthalenediisocyanate. The material obtained via polymerization of a diisocyanate monomer with a diamine monomer is a material (polyurea).

It is preferable that a poling treatment of a polymeric piezoelectric film is conducted until polarization reversal occurs, and the polarization reversal is generated by applying a poling electric field while repeatedly reversing the direction of the filed. Sufficient formation of such the polarization distribution depends on temperature. At room temperature, several 10,000-several 100,000 times of reversal may be done, but at a temperature of at least 80° C., several times to several tens of times may be good enough. Further, in the case of a piezoelectric element for reception, a corona treatment at 1 mW/cm²-1 kW/cm² may be conducted at normal pressure during film formation of the element.

PZT is frequently employed as a material of a piezoelectric element for transmission, but the material containing no lead has recently been recommended. Examples thereof include quartz, lithium niobate (LiNbO₃), potassium niobate tantalate [K(Ta, Nb)O₃], barium titanate (BaTiO₃), lithium tantalate (LiTaO₃), strontium titanate (SrTiO₃) and so forth. Further, the material used for a piezoelectric element for reception is a resin having 60-100 mol % of at least one selected from the group consisting of polyvinylidene fluoride, polyurea, polyamide, polyimide, polyester and polyolefin.

As shown in FIG. 1 and FIG. 2, acoustic lens 5 may be put together with second acoustic matching layer 4 to converge the ultrasound wave. The matching layer also has a double layer structure in this case, but in some objects, a multilayer structure may be accepted, and a single layer structure or a structure having no matching layer may also be accepted. The number of matching layers is preferably 2-3.

### EXAMPLE

Next, the present invention will be explained employing examples, but the present invention is not limited thereto.

### Example 1

### Preparation of a Three Layer Type Acoustic Matching Later Provided Between a Piezoelectric Element for Transmission and a Piezoelectric Element for Reception

PZT was utilized for the piezoelectric element for transmission. The following organic material was also utilized for the piezoelectric element for reception. A piezoelectric element for reception with 82 mol % of polyvinylidene fluoride in (polyvinylidene fluoride/trifluoroethylene) was designated as Sample 1, and a piezoelectric element for reception with 87 mol % of polyvinylidene fluoride in (polyvinylidene fluoride/trifluoroethylene) was designated as Sample 2. Further, a piezoelectric element for reception with 87 mol % of polyvinylidene fluoride in {polyvinylidene fluoride/perfluoroalkylvinyl ether(PFT)} was designated as Sample 3. Then, arraying was conducted for each of these samples to obtain a signal corresponding to the sample via a commonly known method. Epoxy resin AW-106/HV953U (produced by Nagase Chemtex Corporation) was employed as a resin for an ultrasound probe of the present invention as shown in FIG. 2, and 12.5 times in quantity of tungsten powder having an average particle diameter of 6-8 μm, with respect to the above-described AW resin, was charged into the resin for the first layer provided on a piezoelectric element for reception 2 of the ultrasound probe of the present invention in FIG. 2. For the second layer, 3.6 times in quantity of tungsten powder having an average particle diameter of 3-5 μm, with respect to the above-described AW, was charged into the resin. The third layer had only the above-described AW resin, and filler was not added into the third layer. Cross-linkage of the resin was conducted at 100° C. for 10 minutes. Under the foregoing condition, the first layer was formed by using a resin solution of the first layer, the second layer was formed by using a resin solution of the second layer, and the third layer was formed by using a resin solution of the third layer. Each layer had a thickness of 75 μm, and a gradient type laminated acoustic matching layer having a total thickness of 225 μm was formed.

The resulting gradient type laminated acoustic matching layer had the following characteristics. That is, the acoustic impedance of the first layer, the acoustic impedance of the second layer and the acoustic impedance of the third layer were Z=14.8 Mrayl, Z=6.2 Mrayl and Z=5 Mrayl, respectively. It was confirmed that the acoustic impedance value varied gradually from approximately 28 Mrayl of acoustic impedance of a piezoelectric element to 1.5 Mrayl of acoustic impedance of a human body to form a matching layer.

In this way, harmonic imaging having a high S/N ratio (S/N ratio of Sample 1<S/N ratio of Sample 2=S/N ratio of Sample 3) was realized by providing an acoustic impedance between a piezoelectric element for transmission and a piezoelectric element for reception.

As is clear from the above-described, it is to be understood that the ultrasound probe of the present invention is an ultrasound probe exhibiting high acoustic performance.

Accordingly, in the case of the ultrasound probe of the present invention, S/N ratio and sensitivity can be improved by providing an acoustic impedance between a piezoelectric element for transmission and a piezoelectric element for reception.

### EFFECT OF THE INVENTION

The present invention can provide a multichannel array type ultrasound probe comprising a transmission-reception separation type composite piezoelectric element, in which the operation during transmission and reception of ultrasound wave is separated, wherein a piezoelectric element for transmission comprises a ceramic material, a piezoelectric element for reception comprises a high sensitivity organic piezoelectric element material, and an acoustic matching layer is provided between a piezoelectric element for transmission and a piezoelectric element for reception to enhance high sensitivity in S/N ratio.

What is claimed is:

1. A multichannel array type ultrasound probe comprising a transmission-reception separation type composite piezoelectric element,
wherein the composite piezoelectric element comprises an acoustic matching layer provided between a piezoelectric element for transmission comprising a ceramic material and a piezoelectric element for reception comprising an organic material.

2. The multichannel array type ultrasound probe of claim 1,
wherein the organic material comprises a resin selected from the group consisting of polyvinylidene fluoride, polyurea, polyamide, polyimide, polyester and polyolefin in an amount of 60-100 mol %.

3. The multichannel array type ultrasound probe of claim 1,
wherein the organic material further comprises a vinylidene fluoride-perfluoroalkylvinyl ether copolymer or a vinylidene fluoride-perfluoroalkoxyethylene copolymer, provided that a content of the vinylidene fluoride is 85-99 mol %, and a content of the perfluoroalkylvinyl ether or the perfluoroalkoxyethylene is 1-15 mol %.

4. The multichannel array type ultrasound probe of claim 1,
wherein the acoustic matching layer comprises plural layers, and an acoustic impedance value of each of both outermost layers among the plural layers is 5-28 Mrayl.

5. The multichannel array type ultrasound probe of claim 1,
wherein the acoustic matching layer comprises a plurality of layers each having a different filler content.

6. The multichannel array type ultrasound probe of claim 5,
wherein the filler is least one selected from the group consisting of tungsten, ferrite and aluminum.

7. The multichannel array type ultrasound probe of claim 1,
wherein the ceramic material comprises one selected from the group consisting of PZT, quartz, lithium niobate (LiNbO₃), potassium niobate tantalate [K(Ta,Nb)O₃], barium titanate (BaTiO₃), lithium tantalate (LiTaO₃) and strontium titanate (SrTiO₃).

8. The multichannel array type ultrasound probe of claim 1,
wherein the acoustic matching layer comprises a binder selected from the group of consisting of polyvinyl butyral, polyolefin, polycycloolefin, polyacrylate, polyamide, polyimide, polyester, polysulfone, silicone, epoxy and derivatives thereof.

9. The multichannel array type ultrasound probe of claim 1,
wherein the transmission-reception separation type composite piezoelectric element comprises the piezoelectric element for transmission and the piezoelectric element for reception, provided between an acoustic lens and a backing layer.

10. A method of manufacturing the multichannel array type ultrasound probe, comprising the step of:
coating the acoustic matching layer of claim 1 via ink-jet.

11. The method of claim 10, further comprising the step of:
discharging a liquid comprising a binder or a filler as ink used for the ink-jet.
